(19) United States
(12) Patent Application Publication
(10) Pub. No.: US 2005/0019765 A1
(43) Pub. Date: Jan. 27, 2005

(54) Methods for treating disorders of the nervous and reproductive systems

(76) Inventors: Cheryl L Wellington, Vancouver (CA); Angela R. Brooks-Wilson, Richmond (CA); Michael R. Hayden, Vancouver (CA)

Correspondence Address: CARELLA, BYRNE, BAIN, GILFILLAN, CECCHI,;STEWART & OLSTEIN, 5 BECKER FARM ROAD, ROSELAND, NJ, 07068, US

(21) Appl. No.: 10/479,879

(22) PCT Filed: Jun. 7, 2002
(86) PCT No.: PCT/CA02/00836

Related U.S. Application Data
(60) Provisional application No. 60/297,102, filed on Jun. 8, 2001.

(51) Int. Cl. C12Q 1/68; A61K 49/00

(52) U.S. Cl. 435/6; 424/9.2

(57) ABSTRACT

The invention features methods for treating, preventing or modulating a neurological disease or disorder, or for modulating an anaesthetic or a fertility process by administering compounds that modulate ABCA1 expression or activity. The invention also features methods for identifying compounds useful for such methods.

This application claims priority of U.S. Provisional Application No. 60/297,102, filed 8 Jun. 2001, the disclosure of which is hereby incorporated by reference in its entirety.

### FIELD OF THE INVENTION

The present invention relates to the field of treating, preventing and/or modulating neurological diseases and disorders, modulating anaesthetic and/or fertility processes using compounds that modulate ABCA1 expression and/or activity as well as screening assays for identifying compounds useful in these methods.

### BACKGROUND OF THE INVENTION

Regulation of lipid homeostasis is an essential process for many cellular and bodily functions, and is controlled to a large extent by the regulated transport of cholesterol and phospholipids carried on lipoproteins. Almost all of the lipoproteins are formed in the liver, which is where most of the plasma cholesterol, phospholipids and triglycerides (except those obtained from dietary sources) are synthesized. Large quantities of phospholipids and cholesterol are present in both the plasma and internal cellular membranes of all cells, and the ratio of cholesterol to phospholipids is a critical determinant of membrane fluidity. Formation of membranes is critically dependent on the transport of the appropriate lipids in the form of lipoprotein particles.

The primary function of the lipoproteins is to transport their lipid components in the blood. Aberrant regulation of lipid and lipoprotein homeostasis can lead to a variety of diseases including atherosclerosis. Major risk factors for atherosclerosis include elevated plasma LDL-cholesterol (LDL-C) and depressed HDL-cholesterol (HDL-C) levels. ABCA1 has recently been identified as a key regulator in the formation of HDL particles, acting as a crucial transporter of cholesterol and phosopholipids from cells to ApoA1. Mutations in the ABCA1 gene have been identified as the underlying cause of Tangier Disease (TD) and of a dominantly inherited form of hypoalphalipoproteinemia (FHA) associated with greatly reduced efflux of cholesterol and phospholipid to ApoA1. TD is a rare form of genetic HDL-C deficiency in which patients are homozygous or compound heterozygous for mutations in both alleles of ABCA1, resulting in nearly undetectable ApoA1-mediated cholesterol efflux and virtually no circulating HDL-C. The inability to remove excess cholesterol from peripheral tissues of TD patients often results in clinical manifestations of orange tonsils, hepatosplenomegaly, peripheral neuropathy, and premature coronary artery disease (CAD). TD patients are also observed to have small families, suggesting that aspects of reproductive fitness may be an additional effect of mutations with in ABCA1. Persons heterozygous for ABCA1 mutations have FHA, a less severe clinical outcome of deficient cholesterol efflux characterized with circulating HDL-C levels at or below the 5^{th} percentile for an age- and sex-matched population.

ABCA1 belongs to the ATP-Binding-Cassette family of transporters and is a member of the full-size class A group of transporters. This highly conserved family of proteins mediates the ATP-dependent unidirectional transmembrane transport of numerous substrates. Full-size transporters consist of tandemly arranged ATP binding cassettes and two transmembrane domains consisting of 12 membrane spanning segments, whereas half-size transporters form dimers each containing one ATP-binding cassette and one transmembrane domain. ABC transporters have been localized to the plasma membrane, Golgi apparatus, endoplasmic reticulum, peroxisomes, and intracellular secretory vesicles. The vital role ABC transporters play in transport is underscored by the many human diseases resulting from ABC mutations including cystic fibrosis, Stardgardt disease, retinitis pigmentosa, cone-rod dystrophy, age-related macular degeneration, progressive familial intrahepatic cholestasis, Dubin-Johnson syndrome, adrenoleukodystrophy, and pseudoxanthoma elasticum.

ABCA1 generates HDL by transferring cholesterol onto apoprotein-A1 (apoA1) in a process known as cholesterol efflux, which is also an essential component of male fertility. Sperm are made in the testes and released into the epididymis, where they await ejaculation into the female reproductive tract. Although differentiated, epididymal sperm are not capable of fertilization and must first complete their maturation in the female reproductive tract. This maturation process is known as capacitation and is known to require efflux of cholesterol from the sperm plasma membrane. How this occurs at the molecular level is not well understood. The present invention relies in part on the discovery that ABCA1 is highly expressed in the testes (being present in Sertoli and Leydig cells as well as in differentiating spermatids). ABCA1-deficient mice develop highly vacuolated and largely aspermic seminiferous tubules, indicating that ABCA1 is necessary for normal spermatogenesis. In accordance with the present invention, ABCA1 is also present in epididymal sperm where it can act to transport cholesterol during capacitation. In vivo, follicular HDL is an endogenous cholesterol acceptor for capacitating sperm, and HDL can stimulate capacitation in vitro. In accordance with the present invention, because cholesterol efflux through ABCA1 is dependent upon apoA1 the ability of apoA1 to stimulate capacitation of murine sperm in vitro was tested using a chlortetracycline assay. The results showed that de-lipidated apoA1 capacitates sperm in a dose-dependent manner, thereby showing ABCA1 to be a key transporter of cholesterol for capacitating sperm implicating it as a molecular link between cholesterol metabolism in the periphery and reproductive systems.

The brain is the most cholesterol rich organ in the body, underscoring the essential nature of lipid homeostasis for brain physiology. Neuronal function in both the central and peripheral nervous systems depends heavily on appropriate regulation of membrane structure and composition. Membranes are needed for the plasma membrane itself, formation of synaptic vesicles, vesicular transport, and endocytosis and exocytosis of vesicles. As well, lipids are salvaged and recycled during synaptic remodeling, following neuronal lesions including neuroinflammatory reactions, and the ability to uptake and efflux lipids plays very important roles in these processes. The neuronal plasma membrane is highly compartmentalized, and specific regions of the membrane have very different protein compositions and functions, and it is not unreasonable to propose that the lipid composition of the different portions of the neuronal plasma membrane may vary as well. Virtually all functions of the neuron depend on membranes at some level. The high level of ABCA1 in the brain is consistent with multiple roles in aspects of lipid and lipoprotein metabolism in the brain.

Because the brain and testis have highly developed membrane systems with functions requiring a tightly regulated membrane environment, the development of drugs for the treatment of infertility and neurological disorders has so far progressed slowly. The present invention solves this problem by identifying ABCA1 as a key molecule in regulating essential neurological- and fertility-related processes, thereby providing a means of screening agents for ability to advantageously affect such processes while simultaneously limiting the pool of compounds to be screened to those already known to be modulators of ABCA1.

### BRIEF SUMMARY OF THE INVENTION

In one aspect, the present invention relates to a method for identifying an agent useful in treating an ABCA1-dependent neurological condition comprising:
(a) administering to an animal exhibiting said neurological condition an agent that modulates ABCA1 biological activity, and
(b) detecting a beneficial change in said neurological condition in said animal following said administering and compared to when said agent is not administered,
thereby identifying an agent useful in treating said ABCA1-dependent neurological condition.

Such an agent is selected from those that either enhance or inhibit ABCA1 biological activity. In preferred embodiments, the ABCA1 biological activity affected by such agent may include ABCA1-gene expression and be some activity of the ABCA1-polypeptide, such as HDL-cholesterol transport, ion transport, ATP binding, ATP hydrolysis, and/or phospholipid transport. In other preferred embodiments, this may include ABCA1 biological activity that is due to a change in stability of ABCA1 polypeptide, a change in ABCA1 membrane insertion and a change in ABCA1 membrane channel formation.

In other preferred embodiments, the neurological condition being screened for is a neurological condition of the central nervous system or of the peripheral nervous system and such neurological condition may be characterized by neuronal loss or dysfunction. In specific embodiments, the neurological condition is one or more of Alzheimer's Disease, dementia pugilistica, Parkinson's Disease, Huntington's Disease, Niemann-Pick disease, multiple sclerosis, a neuropathy and an ischemic condition.

In a preferred embodiment, where the neurological condition involves an ischemic condition, this ischemic condition may be of the central, peripheral, or compression type. In specific embodiments, this ischemic condition is one of stroke and/or cerebral artery infarction.

In other preferred embodiments, the neurological condition is caused by a defect in myelin repair or production, especially where said defect in myelin repair or production is caused by a process selected from demyelination, the removal of myelin debris following injury and remyelination.

In another aspect, the present invention relates to a method of treating a neurological condition comprising administering to an animal afflicted with such condition an effective amount of an agent exhibiting beneficial activity using a screening method as disclosed herein, with similar embodiments as described herein. In one preferred embodiment, the agent used for such treatment is one that was initially identified as having beneficial activity using a screening method as disclosed herein.

In an additional aspect the present invention relates to a method for identifying an agent useful in regulating fertility in a mammal comprising:
(a) administering to a mammal an agent that modulates ABCA1 biological activity, and
(b) detecting a change in fertility of said mammal following said administering and compared to when said agent is not administered,
thereby identifying an agent useful in regulating fertility of said mammal.

In specific embodiments, such agent may inhibit or enhance the activity of ABCA1 biological activity, wherein the latter may include ABCA1-gene expression or activities of the ABCA1-polypeptide, such as HDL-cholesterol transport, ion transport, ATP binding, ATP hydrolysis, and/or phospholipid transport, and also may include agents that modulate other activities of ABCA1, such as a change in stability of ABCA1 polypeptide, a change in ABCA1 membrane insertion and a change in ABCA1 membrane channel formation.

Such agents may have the effect of either increasing or decreasing fertility in an animal, especially a mammal, most especially a human being. In one such embodiment, the mammal is male and ABCA1-modulating agent has the effect of increasing spermatogenesis and/or capacitation of one or more sperm cells of this male and thereby increase fertility. In a preferred embodiment of the latter effect, the agent is a positive modulator of one or more of the functions of ABCA1 genes or proteins and thus has the effect of enhancing ABCA1-biological activity.

In another such embodiment, the mammal is male and the agent decreases spermatogenesis and/or capacitation of one or more sperm cells of this male, thereby reducing fertility. In a preferred embodiment of the latter effect, the agent is a negative modulator of one or more of the functions of ABCA1 genes or proteins and thus has the effect of decreasing ABCA1-biological activity.

In another such embodiment, the agent decreases capacitation of one or more sperm cells collected from a male mammal, thereby protecting said sperm cells from the long-term decrease in fertilization potential by cryopreservation.

In another such embodiment, the agent increases capacitation of one or more cyropreserved sperm cells originating from a male mammal, thereby increasing the fertilization potential of cryopreserved sperm.

In another such embodiment, the mammal is female and the agent may increase fertility or decrease fertility. In one such embodiment, the ABCA1-modulating agent promotes implantation into the endometrium, thereby increasing fertility. In a preferred embodiment of the latter process, the agent is a positive modulator of ABCA1 gene expression or polypeptide activity and thus inhibits ABCA1-biological activity.

In another such embodiment, the ABCA1-modulating agent inhibits implantation in the endometrium, thereby decreasing fertility. In a preferred embodiment of the latter process, the agent is a negative modulator of ABCA1 gene expression or polypeptide activity and thus inhibits ABCA1-biological activity.

In a further aspect, the present invention relates to a method for identifying an agent useful in regulating fertility in a male mammal comprising:
(a) contacting a sperm cell of said mammal with an agent that modulates ABCA1 biological activity and under conditions promoting said contacting and supporting viability of said sperm cell, and
(b) detecting a change in the ability of said sperm cell to fertilize an ovum of a mammal of the same species as compared to when said sperm cell is not so contacted,
thereby identifying an agent useful in regulating fertility of said male mammal.

In specific embodiments thereof, the agent may inhibit ABCA1 biological activity or may enhance ABCA1 biological activity, which activity may include gene expression or any of the functions of the ABCA1-polypeptide, including HDL-cholesterol transport, ion transport, ATP binding, ATP hydrolysis, and/or phospholipid transport, or may include effects on stability of ABCA1 polypeptide, a change in ABCA1 membrane insertion and a change in ABCA1 membrane channel formation.

Such agents may increase fertility by increasing the ability of said sperm cell to fertilize said ovum. In a specific embodiment, this increase is due to an increase in capacitation of the sperm cell(s), such as where the agent is a positive modulator of ABCA1-biological activity.

Alternatively, the agent may decrease the ability of said sperm cell to fertilize said ovum, especially where this decrease is due to a decrease in capacitation of said sperm cells, such as where the agent is a negative modulator of ABCA1 biological activity.

In a still further aspect, the present invention relates to a method for regulating fertility in a mammal comprising administering to said mammal an agent having fertility-regulating ability in one or more of the screening assays disclosed herein, especially where said agent was first identified as having such physiological activity using said screening assay.

Where said mammal is male, the agent will preferably modulate fertility by modulating the fertility of the sperm cells of said male mammal, especially where the agent has the effect of regulating capacitation of the sperm cells of said male mammal.

Where the mammal is female, the agent preferably modulates fertility by modulating implantation into the endometrium of said female mammal.

In a still further aspect, the present invention relates to a method for modulating the ability of a sperm cell to fertilize an ovum of an animal of the same species as said sperm cell comprising contacting said sperm cell with an agent that exhibits fertility-regulating ability using a screening method as disclosed herein. In a preferred embodiment, said modulation is an increase in the ability of said sperm cell to fertilize said ovum, especially where this agent acts to promote capacitation of said sperm cells. In an alternative embodiment, said modulation is a decrease in the ability of the sperm cell to fertilize the ovum, especially where this agent acts to inhibit, or prevent, capacitation of said sperm cells.

In a yet still further aspect, the present invention provides a method of treating adverse conditions related to fertility processes, such as adverse conditions arising from pregnancy, especially conditions such as preeclampsia, involving such processes as hypertension and edema resulting from, or related to, pregnancy.

In a preferred embodiment of such processes, the present invention provides a method of treating preeclampsia in a mammal comprising administering to a mammal afflicted therewith an effective amount of an agent that exhibits fertility-regulating ability using a method of claim 28-43.

In another such preferred embodiment, the present invention relates to a method of preventing preeclampsia in a mammal comprising administering to a mammal at risk thereof an effective amount of an agent that exhibits fertility-regulating ability using a screening method disclosed herein, most preferably where said method was used to first identify such agent as having this activity. In a preferred embodiment, the mammal to be treated is a human being.

The present invention also relates to such screening methods wherein the identification of such an agent is itself a step of the procedure. In all of the screening methods of the invention, which may be either in vitro or in vivo methods, the ABCA1-modulating agent may or may not have been previously shown to modulate ABCA1-biological activity prior to use in the screening method. Thus, a compound may be screened that was not previously known to be such a modulator but the method of determining its ability to modulate ABCA1 may itself be part of the screen.

Thus, the present invention also relates to a method of treating an animal for an ABCA1-dependent neurological condition comprising administering to an animal afflicted with such condition an effective amount of an ABCA1-modulating agent, preferably where the agent has activity using a screening method of the invention, most preferably where the agent was first identified as useful in treating said neurological condition using such screening method. In other preferred embodiments, the ABCA1-modulating agent used in the treatment had been previously shown to have ABCA1-modulating activity. Alternatively, it may not have been previously shown to have such activity.

In a further aspect, the present invention relates to a screening method for identifying an agent useful in negating the malfunctioning of a nervous system cell comprising:
(a) contacting a malfunctioning nervous system cell, wherein said malfunctioning promotes the presence of said neurological condition, with an ABCA1-modulating agent under conditions promoting said contacting and otherwise supporting the normal functioning of said cell,
(b) determining a beneficial change in one or more functions of said cell after said contacting wherein said beneficial change is not determined when said contacting does not occur, and
thereby identifying an agent useful in negating malfunctioning of a neurological condition.

The present invention also relates to a screening method for identifying an agent useful in treating an ABCA1-dependent neurological condition comprising:
(a) contacting a malfunctioning nervous system cell, wherein said malfunctioning promotes the presence of said neurological condition, with an ABCA1-modulating agent under conditions promoting said contacting and otherwise supporting the normal functioning of said cell,
(b) determining a beneficial change in one or more functions of said cell after said contacting wherein said beneficial change is not determined when said contacting does not occur, and
thereby identifying an agent useful in treating said neurological condition.

The present invention also relates to a screening method for identifying an agent useful in promoting myelin production in a connective tissue cell whose normal function includes myelin production, comprising:
(a) contacting said connective tissue cell with an ABCA1-modulating agent under conditions promoting said contacting and otherwise supporting myelin production by said cell,
(b) determining an increase in myelin production by said cell after said contacting wherein said increase is not determined when said contacting does not occur, and
thereby identifying an agent useful in promoting myelin production by said cell.

In a preferred embodiment of the latter method the cell of step (a) is deficient in myelin production, such as where the cell is found in the central nervous system or the peripheral nervous system, most preferably wherein said cell is a Schwann cell of an oligodendrocyte. In other preferred embodiments, the contacting occurs in vitro or occurs in vivo.

In a preferred embodiment of any of these screening methods, the ABCA1-modulating agent may have been previously shown to modulate ABCA1-biological prior to use in the screening method. Conversely, the agent may be novel and not previously shown to do so.

The present invention preferably relates to a method of treating a neurological condition in an animal comprising administering to an animal afflicted with said condition an effective amount of an agent first identified as having therapeutic activity using a screening method of the invention.

In another aspect, the present invention relates to a method for identifying an ABCA1-related cause of reduced fertility in a male patient afflicted with said reduced fertility comprising identifying in one or more sperm cells from said patient a reduced ABCA1-biological activity relative to a sperm cell from a patient without said ABCA1-related infertility.

In a preferred embodiment, the reduced ABCA1-biological activity is a decrease in activity of an ABCA1-polypeptide in said one or more sperm cells, and/or a decreased amount of ABCA1-polypeptide in said one or more sperm cells, and/or decreased expression of an ABCA1 gene in said one or more sperm cells and/or the decreased expression is due to a polymorphism in a promoter or other non-coding region of said ABCA1 gene and/or the reduced ABCA1-biological activity is due to a polymorphism in a coding region of an ABCA1 gene in said one or more sperm cells.

The present invention also relates to a method for identifying a male patient afflicted with reduced fertility as a candidate for treatment of said reduced fertility using an ABCA1-modulating agent comprising identifying in said male patient a reduced amount of ABCA1-biological activity using a screening method of the invention. In separate embodiments, the ABCA1-modulator is a positive modulator of ABCA1-biological activity and/or was previously shown to modulate ABCA1-biological activity.

The present invention still further relates to a method of preventing capacitation of sperm cells during freezing and/or cryopreservation comprising preserving sperm cells by freezing/storing in a composition comprising a modulator, preferably a negative modulator, of ABCA1 biological activity, thereby preventing capacitation during such cryopreservation.

The present invention yet further provides a method for facilitating processes requiring sperm fertility, such as in vitro fertilization, comprising addition of an ABCA1 modulator, preferably a positive modulator, to a sample of sperm cells either not capacitated, or inadequately capacitated, or known to be comprised as to capacitation, preferably following some form of cryopreservation, or other freezing and/or storage process, thereby enhancing and/or restoring the capacitation status of said sperm cells.

### Definitions

As used herein, the following terms have the indicated meaning unless expressly stated otherwise.

By “neurological disease” is meant any progressive neurodegenerative disease of the central, peripheral, or autonomic nervous system that is characterized by neuronal loss or dysfunction, including but not limited to Alzheimer's Disease, dementia pugilistica, Parkinson's Disease, Huntington's Disease, Niemann-Pick disease, multiple sclerosis, neuropathies (e.g. central, peripheral, compression type) and ischemic conditions such as stroke and cerebral artery infarction. Defects in myelin repair and/or production are also considered neurological diseases. The defects may arise during the process of demyelination, the removal of myelin debris following injury, or the remyelination process.

By “neurological disorder” is meant any physical injury to the central, peripheral or autonomic nervous system. The injury can arise from trauma or surgery.

By “neurological condition” is meant any neurological disease or disorder.

By “anaesthetic process” is meant any treatments that act through neuronal membranes or receptors, but especially through membrane compositions or cholesterol/phospholipid balance or membrane/protein interactions, to effect anesthesia.

By “fertility process” is meant any step directly involved in the process of gametogenesis, fertilization, conception, implantation or embryogenesis.

By “modulation of a fertility process” is meant any effect that alters the fertility of the subject. Modulation of a fertility process in an inhibitory manner can result in contraception. Enhancement of a fertility process can result in an increased likelihood of fertilization and embryogenesis and can be done for the purpose of overcoming a reproductive defect. This enhancement can be effected by increasing the potency of the sperm, by causing the endometrium to become more receptive to embryo implantation, or by reducing the probability that the uterus will expel, absorb or otherwise reject an implanted embryo.

By “reproductive defect”, is meant any cause of infertility in either a male or a female. A “reproductive defect” can include a defect in sperm capacitation, spermatogenesis, oogenesis, fertilization, failure of an embryo to implant in the endometrium or spontaneous abortion after embryo implantation, and related conditions. This includes any defect in part of the process of sperm-oocyte fusion and activation of development and division of the fertilized ovum. It can include defects in placental development which prevent or reduce normal fetal development, and more generally, any disorder that involves membrane malfunction in the developing embryo.

By “contraception” is meant a temporary and reversible method for reducing fertility. Contraception can be induced in a male by rendering the sperm incapable of fertilizing an ovum, or in a female by either preventing the ovum from becoming fertilized, preventing a fertilized embryo from implanting in the endometrium, or causing the uterus to expel, absorb or otherwise reject an implanted embryo.

By “polypeptide” is meant any chain of more than two amino acids, regardless of post-translational modification such as glycosylation or phosphorylation.

By “regulatory region” is meant a region that, when operably linked to a promoter and a gene (e.g., a reporter gene), is capable of modulating the expression of the gene from the promoter. Regulatory regions include, for example, nuclear hormone transcription factor binding sites such as those described herein and may be found in intronic sequence.

By “promoter” is meant a minimal sequence sufficient to direct transcription of an operably-linked gene.

By “modulates” or “modulation” is meant increase or decrease, and related concepts. Modulation of a human condition such as fertility includes increasing or improving fertility (as in the case of persons with reproductive defects) or decreasing or reducing fertility (as in the case of persons seeking pre- or post-fertilization contraception). Modulation of the biological activity of a protein or gene means increasing or decreasing measurable activities of the protein or gene. Those skilled in the art are familiar with an extremely broad range of measurable activities for proteins or genes, many or all of which can be modulated in various ways. In this specification, “modulates” is also used in the sense that preferably, a compound that modulates specific biological activities of ABCA1 protein or gene, such as LXR-mediated transcription, RXR-mediated transcription, or ABCA1 gene expression, does so by at least 5%, more preferably by at least 10%, and most preferably by at least 25% or even at least 50%.

By “purified antibody” is meant antibody which is at least 60% by weight, free from proteins and naturally occurring organic molecules with which it is naturally associated. Preferably, the preparation is at least 75%, more preferably 90%, and most preferably at least 99%, by weight, antibody. A purified antibody may be obtained, for example, by affinity chromatography using recombinantly-produced protein or conserved motif peptides and standard techniques. A preferred antibody binds to the ABCA1 polypeptide sequence.

By “specifically binds” is meant an interaction between a molecule and its target or binding partner (e.g. an antibody and its antigenic substrate) that has a substantially higher affinity and specificity than the interaction between the molecule and other, non-target molecules of the sample.

By “polymorphism” is meant that a nucleotide or nucleotide region is characterized as occurring in several different forms. A “mutation” is a form of a polymorphism in which the expression level, stability, function, or biological activity of the encoded protein is substantially altered.

By “LXR” is meant nuclear receptors LXR and LXR. Preferred LXRs include human LXR (GenBank accession no. 013133) and human LXR (GenBank accession no. P55055)(see Apfel et al., Mol. Cell. Biol. 14:7025-7035 (1994); Willy et al., Genes Dev. 9:1033-1045 (1995); and Song et al., Proc. Natl. Acad. Sci. USA 91:10809-10813 (1995), each of which is hereby incorporated by reference).

By “RXR” is meant nuclear receptors RXR, RXR, and RXR. Preferred RXRs include human RXR (GenBank accession no. Q13133), human RXR (GenBank accession no. S37781), and human RXR. (GenBank accession no. Q13133).

By “ABC transporter” or “ABC polypeptide” is meant any transporter that hydrolyzes ATP and transports a substance across a membrane. Preferably, an ABC transporter polypeptide includes an ATP Binding Cassette and a transmembrane region. Examples of ABC transporters include, but are not limited to, ABCA1, ABC2, ABCR, and ABC8.

By “ABCA1 polypeptide” is meant a polypeptide having substantial identity to an ABCA1 polypeptide having the amino acid sequence disclosed in WO 01/15676. This protein is the functional full length 2261 amino acid ABCA1 protein. Initially, Luciani et al. (Genomics 21:150-159, 1994) predicted the mouse ABCA1 protein to be 2201 amino acid residues based on nucleotide sequence EMBL Accession No. X75926. No function was known for ABCA1 at that time. Subsequently Langmann et al. described a human ABCA1 protein with 2201 amino acids and a predicted molecular weight of 220 kDa based the cDNA (GenBank accession no. AJ012376). Hayden et al. (U.S. Provisional Patent Application Ser. No. 60/138,048, Jun. 8, 1999) identified the fully functional ABCA1 protein with 2261 rather than 2201 amino acid residues. The correct number of amino acids and the sequence were subsequently confirmed by Pullinger et al. (Biochem. Biophys. Res. Comm. 271, 451-455, 2000) and Santamorina-Fojo et al. (PNAS 97(14): 7987-7992, 2000) GenBank Entrez Protein database Accession No.: AAF86276.

By “ABCA1 biological activity” is meant any measurable biological activity known or available to those skilled in the art. A wide variety of such measurable acitivites are known. Such activities include, for the protein, hydrolysis or binding of ATP, transport of a compound (e.g., cholesterol, phospholipid, interleukin-1), ion across a membrane, regulation of cholesterol or phospholipid levels (e.g., either by increasing or decreasing HDL-cholesterol or LDL-cholesterol levels), activity, binding, expression level, or protein/protein interactions, etc., etc. For the ABCA1 gene, such activities include transcription activity, reporter gene activity, mRNA amount, processing or stability, etc. Biological activity may be measured in a cell-based or a cell free assay system.

By “malfunctioning nervous system cell” is meant a cell derived from nervous tissue, such as a neuron or a glial cell, whose normal functioning, when disrupted, is somehow related to an identifiable neurological condition.

### BRIEF DESCRIPTION OF THE DRAWINGS

FIG. 1 shows expression of ABCA1 in adult FVB murine tissues. Panel A shows expression of ABCA1 mRNA as detected by RT-PCR and expressed relative to 18s rRNA as an internal control (N=5). Panel B shows ABCA1 protein expression using a Western Blot with RAW murine macrophages stimulated with 9-cis-retinoic acid and 22-R-hydroxycholesterol as a positive control. (N=3) Panel C is a study similar to Panel B but shows expression relative to GAPDH protein on the same membrane (N=3).

FIG. 2 shows several patterns for sperm based on state of capacitation (Panel A), the results of a chlortetracycline assay for capacitation mediated by either BSA or ApoA1 (Panel B) and tissue distribution of ABCA1 (Panel C).

### DETAILED DESCRIPTION OF THE INVENTION

This invention relates to the discovery that the ABCA1 protein in mammals is intimately involved in certain neurological disease processes and certain processes required for fertility. As such, ABCA1 is an important target for therapeutic agents which can be used to treat or modulate these ABCA1-linked conditions.

The Role of ABCA1 in the Nervous System

Tangier Disease patients are known to have peripheral neuropathy associated with lipid accumulation in Schwann cells. Nerve loss appears to precede lipid loading of the Schwann cell. This finding suggests that ABCA1 may be important for normal neuronal function and deficiency of ABCA1 results in two outcomes: increased susceptibility of neurons to death and Schwann-cell accumulation of lipids acquired from dead neurons.

Because myelin sheaths are primarily composed of lipids, the demyelinating disorders are of interest with respect to ABCA1 biology. Multiple sclerosis is the most well known of the demyelinating disorders and affects neurons within the CNS. There are several potential levels where ABCA1 could play a role in MS and related disorders. First, ABCA1 may have important functions for the oligodendrocyte/Schwann cell that act to maintain the lipid composition of myelin. Second, in parallel with the potential role of ABCA1 in the peripheral neuropathy associated with TD, it is possible that ABCA1 may directly affect the neuron and secondarily affect the oligodendrocyte. Third, due to its important role in macrophage biology, ABCA1 may play a role in neuroinflammation and associated diseases mediated by macrophages (in the PNS) or microglia (in the CNS). Neuroinflammation is an important aspect of MS, as neuronal macrophages (microglia) are thought to mediate an autoimmune reaction against myelin-associated proteins. A deficiency of ABCA1 may also exacerbate neuropathology by interfering with the ability of microglia or macrophages to properly dispose of lipids scavenged from degenerating cells in neuroinflammatory diseases. Conversely, an increase of ABCA1 may help to preserve these functions.

A well-established risk factor for Alzheimer's Disease (AD) is inheritance of the apoE4 allele. How this allele results in increased susceptibility to AD is not known. In AD, there is a shift in the proteolytic cleavage of amyloid precursor protein (APP) toward production of abeta, a fragment of APP that has neurotoxic properties and associates with CNS lipoproteins where it may contribute to their oxidation. Abeta production increases in the presence of a high cholesterol diet, and it is hypothesized that excess cholesterol contributes to dysfunction of many membrane-bound proteases that together increase abeta levels. Statins have recently been shown to be effective in slowing the rate of AD progression, and are thought to retard the rate of abeta deposition by lowering the level of cholesterol in the brain. ABCA1 can use apoE as a cholesterol acceptor, but it is not yet known whether different apoE alleles have different abilities to act as a cholesterol acceptor from ABCA1. Additionally, it is possible that increasing ABCA1 in the brain may permit more efficient removal of excess cholesterol and retard abeta formation. Blockers of ABCA1 have been utilized to prevent amyloid build-up (see, for example, WO 00/24390 (4 May 2000) and WO 98/48784 (5 Nov. 1998).

Huntington Disease (HD) is another progressive neurodegenerative disease caused by expansion of a CAG trinucleotide encoding glutamine in a protein called huntingtin. Proteolytic cleavage is an important part of HD pathogenesis, however, unlike AD, it is not known how many of the proteases that contribute to HD may be membrane bound. However, there is some evidence that excess cholesterol may also be operative in HD, as HD patients seem to have increased levels of plasma total cholesterol. Like AD, it is possible that upregulation of ABCA1 in the brain may facilitate removal of excess cholesterol and retard the pathogenesis of HD. Interestingly, inheritance of the apoE4 allele seems to delay the age of onset of HD. Therefore, there appear to be some important differences between lipid handling between AD and HD.

ABCA1 may act as a transporter of toxic proteins or protein fragments (e.g., APP) out of cells, suggesting a role in amyloid deposition in Alzheimer's. ABCA1 agonists/upregulators may also be useful in the treatment of other, amyloidogenic and non-amyloidogenic diseases of, or acute injury to the nervous system. Apolipoprotein E is known to have an involvement in Alzheimer's Disease (see: Poirier J. Apolipoprotein E and Alzheimer's disease. A role in amyloid catabolism. Ann N Y Acad. Sci. 924:81-90 (2000).

ABCA1 is expressed in macrophages and is required for engulfment of cells for programmed cell death. The apoptotic process itself and its regulation has important implications for disorders such as cancer, a disease characterized by a failure of cells to undergo cell appropriate death. ABCA1 may facilitate apoptosis, and as such may represent an intervention point for the treatment of non-amyloidogenic diseases of the nervous system or injury to the nervous system including but not limited to Parkinson's Disease, Huntington's Disease, Niemann-Pick Disease, multiple sclerosis, adrenoleukodystrophy, adrenomyeloneuropathy, other neuropathies (ie. diabetic neuropathy) or acute nervous system injury (e.g. traumatic brain or spinal cord injury). Reducing ABCA1 expression or activity or otherwise down-regulating ABCA1 by any method may constitute a treatment for these diseases and injuries by decreasing apoptosis and thus potentially reducing the amount of neuronal cell loss. ABCA1 could, therefore, be used in a method for identification of compounds for use in the treatment of nervous system disease and injury.

Defective intracellular cholesterol transport results in peripheral neuropathy, corneal opacities, and deposition of cholesterol esters in the rectal mucosa, among other maladies. The present invention provides methods of screening modulators of ABC activity for their ability to remedy these disadvantageous results of defective cholesterol transport.

The present invention also provides methods for treating such maladies and for achieving other beneficial effects by using such ABC modulating agents, which may include proteins as well as small organic molecules. Thus, such agents find use in treating neurological diseases or disorders, or for modulating anaesthetic or fertility processes; including treating and/or preventing adverse effects of a variety of nervous system disorders, including amyloidogenic and non-amyloidogenic neurodegenerative diseases and traumatic injury; as well as processes relating to fertility, both male and female.

The invention takes advantage of the identification of ABCA1 protein and mRNA expression in the brain, testis and placenta; tissues not known to be involved in regulation of HDL-C homeostasis or involved in reverse cholesterol transport. For example, localization of ABCA1 to the neuronal and glial cells implicates these transporters in the maintenance of the highly developed membrane systems which characterize the nervous system. ABCA1-dependent cholesterol transport may be vital for membrane integrity and fluidity. The presence of ABCA1 in sperm cells allows this transporter to facilitate cholesterol efflux from the plasma membrane, a critical step in the process of sperm capacitation that ultimately facilitates fertilization.

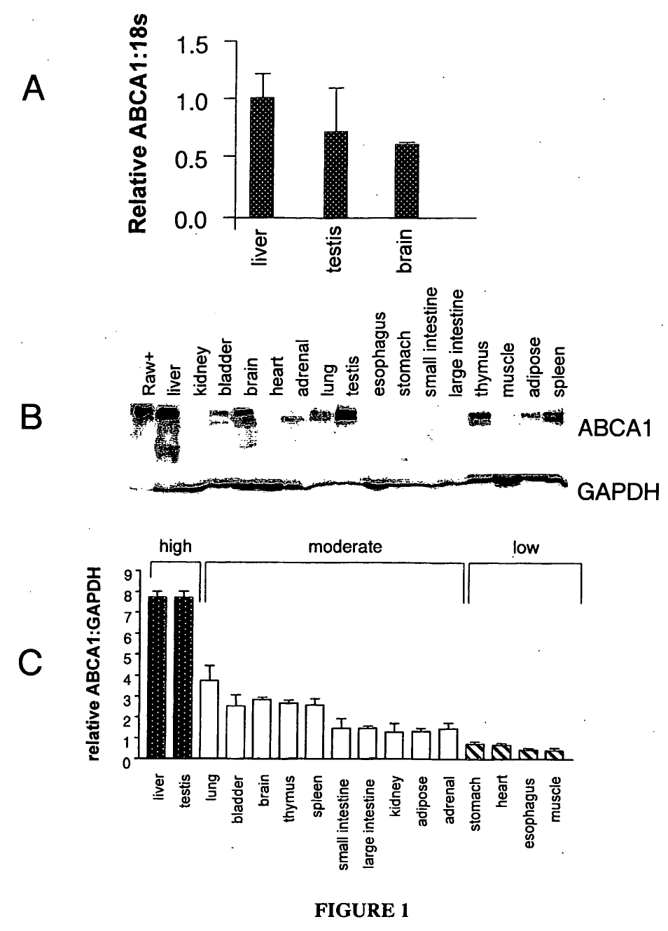

RT-PCR was used to determine the distribution pattern of murine ABCA1 mRNA expression. ABCA1 mRNA was abundant in the brain and testes (FIG. 1) in addition to the placenta. Tissue-specific expression of ABCA1 protein was confirmed using either a novel and specific C-terminal polyclonal antibody or a novel and specific C-terminal monoclonal antibody. Duplicate Western blots, normalized to GAPDH, from three mice were analyzed for ABCA1 protein. Testes contained high levels of ABCA1 protein, whereas brain and placenta contained moderate amounts. There were no marked differences in the distribution pattern of ABCA1 protein expression in C57/BI6 compared to FVB mice. ABCA1 has been found in a number of diverse tissues (see: Lawn et al., Localization of Human ATP-Binding Cassette Transporter 1 (ABC1) in Normal and Atherosclerotic Tissues, Arterioscler. Thromb. Vasc. Biol., pp. 378-385 (March, 2001—available at www.atvbaha.org), where ABCA1 was found in lung, liver, spleen, testis and the central nervous system. The role of ABCA1 in the testis and neuropathology associated with Tangier Disease is also known (see: Lawn et al (2001) at pp. 383-384). For a summary on ABCA1 protein distribution see Wellington et al, ABCA1 mRNA and Protein Distribution Patterns Predict Multiple Different Roles and Levels of regulation, Laboratory Investigation, 82:272-283 (2002).

Expression of ABCA1 within individual cells from brain and testis was determined by in situ hybridization experiments using a specific N-terminal antisense ABCA1 probe as well as by immunohistochemical analysis using a polyclonal and monoclonal ABCA1 antibody.

Particular neuronal populations have high levels of ABCA1 mRNA and protein. For example, cerebellar Purkinje cells were found to have large amounts of ABCA1 mRNA that is restricted to the cell bodies. High levels of ABCA1 protein were found within Purkinje cell bodies, and staining occurred throughout the Purkinje arbor within the molecular layer of the cerebellum. Although murine cerebellar granular neurons did not show any evidence of ABCA1 expression, cortical and striatal neurons as well as glia contained moderately high levels of ABCA1 mRNA and protein.

In the testes, ABCA1 is found predominately in developing spermatozoa, implicating it in spermatogenesis. The primary function of ABCA1 in the brain and testes is not expected to be HDL-C metabolism for reverse cholesterol transport because they are both separated from the plasma by the relatively tight blood-brain- and blood-testis-barrier, respectively.

The central nervous system consists of many cell types that depend on multiple membrane systems for function. The hallmark function of neurons is to carry out synaptic transmission of signals, which depends on cycles of exocytosis of synaptic vesicles and endocytosis of ligand-bound synaptic receptors. Neuronal axons within the CNS (central nervous system) are ensheathed by myelin, a cholesterol-rich membrane structure generated and maintained by oligodendrocytes in the CNS and Schwann cells within the PNS (peripheral nervous system). Defects in the regulation of membrane structure, composition, and homeostasis are predicted to result in neuronal or glial dysfunction and can result in overt disease or increased susceptibility to disease states. Regulation of cholesterol and phospholipid lipid transport is critical for the maintenance of nervous system membranes and alterations in this transport can serve as the basis for diagnosis or treatment of numerous nervous system disorders.

Mutations in ATP-binding cassette transporter proteins can have important functions in neuronal physiology. For example, adrenoleukodystrophy is a severe demyelinating disorder characterized by a progressive breakdown of the myelin sheath resulting from a defect in a peroxisomal ABC transporter. Modulation of ATP-binding cassette transporters like ABCA1 may also influence the flow of cholesterol between macrophages and Schwann cells (PNS) and microglia and oligodendrocytes (CNS). Facilitating this exchange may promote myelin breakdown during the acute phase of neuronal degeneration or traumatic injury, and then speed remyelination during recovery. Alternatively, ABCA1 may provide a therapeutic intervention point to slow the progress of demyelinating diseases like multiple sclerosis, spinal chord injury and other neurological diseases.

A role for cholesterol metabolism in neuroregeneration is suggested by the identification that ApoE secretion and cholesterol accumulation increases in the regenerating sciatic nerve. Resident and monocyte-derived macrophages are recruited to the injury site and are involved in the phagocytosis of myelin during axonal degeneration following a nerve crush injury. Later, these macrophages secrete significant amounts of ApoE distal to the injury site. Early in the regenerative phase, axonal tips contain a high density of LDL receptors. During axonal regeneration, Apol and ApoA1 accumulate distal to the injury site and macrophages become increasingly cholesterol loaded. Remyelination begins during the second and third weeks after injury and Schwann cells that were depleted of cholesterol stores induce LDL receptor expression and accept cholesterol effluxed from loaded macrophages. During this stage of regeneration, ApoA1 and ApoE are present in the extracellular matrix as cholesterol rich lipoproteins. In the peripheral nervous system, macrophages and Schwann cells participate in a regulated cholesterol transfer to supply the cholesterol required for rapid membrane biogenesis during axonal regeneration and remyelination.

Further evidence for an influence of ABCA1 functions in the nervous system comes from Tangier Disease patients who develop a peripheral neuropathy. It is unclear whether this peripheral neuropathy is due to a neuronal or glial effect, however, the condition is characterized by an accumulation of cholesterol, which could cause a compression neuropathy. Alternatively, changes in membrane fluidity and dynamics may lead to an inability to remove cholesterol that impairs neuronal function (an symptoms of compression neuropathy).

ABCA1 may also have a role in vesicle trafficking and synaptic function by altering the membrane phospholipid-cholesterol ratio. Altered membrane composition can affect processes of exocytosis (neurotransmitter release), endocytosis (neurotransmitter recycling) and receptor internalization. This could impact normal neuronal function and development, learning, memory and emotion, as well as processes of neuronal plasticity and neurodegeneration.

Cholesterol transport within the nervous system provides a point of therapeutic intervention for conditions ranging from neurodegenerative disease to traumatic nervous system injury because of the importance of maintaining the integrity of the extensive membrane structures. Additionally, defects in cholesterol transport could be a contributing factor in the risk and progression of chronic disease conditions such as Parkinson's Disease, Huntington's Disease, Niemann-Pick Disease, multiple sclerosis, adrenoleukodystrophy, adrenomyeloneuropathy, and other neuropathies (e.g., diabetic neuropathy). Therapeutic regulation of these processes can also modulate recovery from traumatic brain injury or traumatic spinal cord injury. In accordance with the present invention, methods of diagnosing and treating these conditions are provided and are based on the discovery of ABCA1 expression in certain neuronal populations and glia.

In accordance with the foregoing, the present invention relates to a method for identifying an agent useful in treating an ABCA1-dependent neurological condition comprising:
(a) administering to an animal exhibiting said neurological condition an agent that modulates ABCA1 biological activity, and
(b) detecting a beneficial change in said neurological condition in said animal following said administering and compared to when said agent is not administered,
thereby identifying an agent useful in treating said ABCA1-dependent neurological condition.

Such an agent is selected from those that either enhance or inhibit ABCA1 biological activity. In preferred embodiments, the ABCA1 biological activity affected by such agent may include ABCA1-gene expression and be some activity of the ABCA1-polypeptide, such as HDL-cholesterol transport, ion transport, ATP binding, ATP hydrolysis, and/or phospholipid transport. In other preferred embodiments, this may include ABCA1 biological activity that is due to a change in stability of ABCA1 polypeptide, a change in ABCA1 membrane insertion and a change in ABCA1 membrane channel formation.

In other preferred embodiments, the neurological condition being screened for is a neurological condition of the central nervous system or of the peripheral nervous system and such neurological condition may be characterized by neuronal loss or dysfunction. In specific embodiments, the neurological condition is one or more of Alzheimer's Disease, dementia pugilistica, Parkinson's Disease, Huntington's Disease, Niemann-Pick disease, multiple sclerosis, a neuropathy and an ischemic condition.

In a preferred embodiment, where the neurological condition involves an ischemic condition, this ischemic condition may be of the central, peripheral, or compression type. In specific embodiments, this ischemic condition is one of stroke and/or cerebral artery infarction.

In other preferred embodiments, the neurological condition is caused by a defect in myelin repair and/or production, especially where said defect in myelin repair is caused by a process selected from demyelination, the removal of myelin debris following injury and remyelination.

In another aspect, the present invention relates to a method of treating a neurological condition comprising administering to an animal afflicted with such condition an effective amount of an agent exhibiting beneficial activity in a screening method as disclosed herein, with similar embodiments as described herein. In one preferred embodiment, the agent used for such treatment is one that was initially identified as having beneficial activity in a screening method as disclosed herein, even though said agent may not have been identified using said screening method. The only requirement is that said agent be capable of modulating ABCA1 activity, even if it is as yet not known to do so. Of course, known ABCA1-modulators are especially preferred for such treatment methods.

The Effect of ABCA1 on Male Fertility

Mammalian spermatogenesis is a complex, multistep process whereby, in the testis, spermatogonia undergo proliferation to form spermatids. The final stage of spermatogenesis in the testes involves a shedding of excess cytoplasm to generate free spermatids that are released into the epididymis. The spermatids undergo the final maturation steps upon ejaculation and release into the female genital tract. This process, known as capacitation, functions to prime the sperm plasma membrane so that it becomes responsive to induction of the acrosome reaction upon contact with the zona pellucida, whereby, the acrosomal contents are released through an exocytotic fusion of the acrosomal membrane with the sperm plasma membrane.

Spermatids contain a structure called an acrosome that contains a battery of enzymes involved in facilitating penetration of mature sperm cells through the zona pellucida of the ovum during fertilization. One of the earliest events in capacitation includes a marked efflux of cholesterol from the sperm plasma membrane to HDL and heparin, both of which are found at high concentrations within the female genital tract.

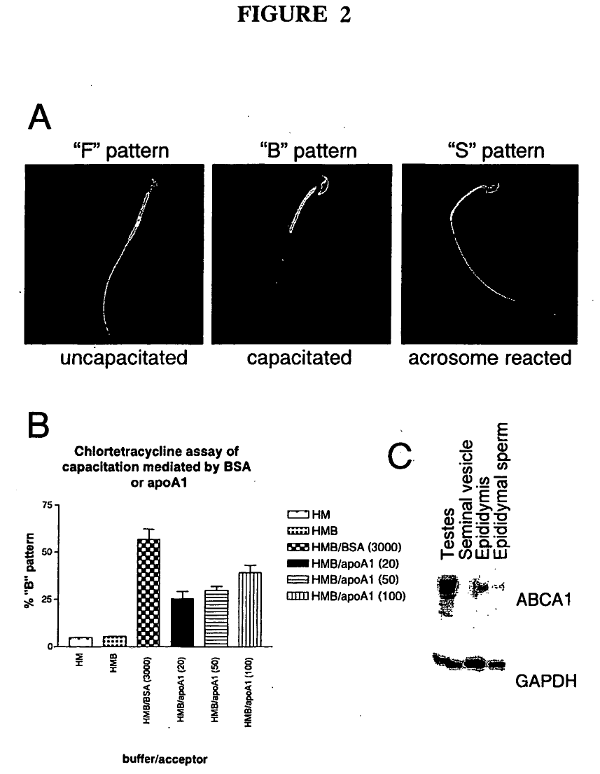

In accordance with the present invention, ABCA1 is expressed in developing sperm. Additionally, ABCA1 is present in sperm collected from the epididymis (FIG. 2). The observation that ABCA1 is expressed on sperm released from the testes provides strong support for a role of ABCA1 in capacitation. Capacitation is known to be dependent on cholesterol efflux, and ABCA1 has a demonstrated role in mediating cholesterol efflux to ApoA1 in the generation of HDL-C particles. Additionally, HDL is present at high concentrations in the female reproductive tract where it acts as a cholesterol and phospholipid acceptor during capacitation.

The process of sperm capacitation provides an advantageous point of therapeutic intervention for regulating male fertility because it is required for fertilization and because sperm are transcriptionally silent and therefore regulation of cholesterol efflux during capacitation must occur at the protein level. Enhancing capacitation by increasing cholesterol efflux through ABCA1 may increase the potency of otherwise compromised sperm. Conversely, inhibiting capacitation by ABCA1 antagonism may result in male contraception by making sperm incapable of penetrating the zona pellucida and effectuating fertilization. As well, increases or decreases in fertilization potential of sperm have applications in long-term storage of cyropreserved sperm for use in in vitro fertilization. Successful cryopreservation of sperm demands that capacitation be inhibited during storage, and that capacitation be facilitated upon thawing and use the preserved sperm. Agents that modulate ABCA1 activity would increase successful sperm storage as well as optimize the fertilizating ability of thawed sperm.

Testes from ABCA1-deficient mice showed distinct vacuolization in a high proportion of seminiferous tubules, suggesting ongoing degeneration (FIG. 2, Panel C). Primary spermatocytes were often observed in the center of the tubule, but few elongated spermatids could be seen. Elongated spermatids were observed in the few tubules that had a more normal morphology. These results suggest that male fertility in ABCA1-deficient mice may be reduced by a loss of spermatid production.

Degenerative, vacuole-laden seminiferous tubules have been noted in several animal models characterized by testosterone deficiency, including aged BDF1 mice (Tanemura, et al., J. Vet. Med. Sci. (1993) 55:703-710), mice following testosterone withdrawal (Kerr, et al., Anat. Rec. (1993) 235:547-549), rats administered a gonadotropin releasing hormone antagonist, and XXY (Klinefelter's syndrome) mice (Lue, et al., Endocrinology (2001) 142:1461-1470). The common morphological features observed in these models and the ABCA1-deficient mice suggest that mice lacking ABCA1 have reduced testosterone levels in their testes that could result from a defect of cholesterol delivery.

Thus, the in vitro fertilization processes used with humans and other animals should be enhanced by the addition of ApoAI, BSPs (or their human functional homologs), or HDL-precursors, or HDL itself. The addition of these ABCA1 interacting components will enhance the necessary cholesterol efflux process for capacitation that precedes the fertilization step.

The Effect of ABCA1 on Female Fertility

The placenta is highly dependent on cholesterol transport for normal function. Like the nervous system, the placenta is characterized by extensive and plastic membrane structures which are critical for normal function. Steroid hormone production is another cholesterol-dependent function of this tissue. The role for ABCA1 function in female fertility is suggested by placental malformations and fetal loss observed in ABCA1-deficient mice (Christiansen-Weber, et al. (2000) 157: 1017-1029). A high incidence of non-productive matings and neonatal death have been reported in these mice and have been attributed to altered steroidogenesis, specifically, reduced estrogen and progesterone levels.

The role of ABCA1 in reproductive dysfunction is not limited to defects of steroidogenesis. Alterations in the membrane environment can affect endometrial receptivity for embryo implantation, a process characterized by marked changes in epithelial morphology. ABCA1 may be required for the regulation and maintenance of basement membrane organization. Subtle alterations in the phospholipid/cholesterol ratio can disrupt maternal/fetal transfer, and may be an underlying cause of preeclampsia. Further evidence suggests that the cholesterol content of the apical plasma membrane increases during early pregnancy following embryo implantation (Murphy et. al., Acta Anat. (Basel) (1987) 128: 76-9).

In accordance with the foregoing, female contraception could be achieved by inhibition of ABCA1 activity, including where ABCA1 inhibitors reduce endometrial receptivity, thereby preventing embryo implantation, or after implantation has occurred, by blocking cholesterol transfer to the apical membrane of uterine epithelial cells. Conversely, compounds that increase ABCA1 activity may promote endometrial receptivity, thereby increasing fertility. Additionally, ABCA1 enhancers can help to maintain pregnancy by facilitating cholesterol transfer to the apical membrane of the uterine epithelial cells as well as preventing or treating preeclampsia by regulating the cholesterol/phospholipid ratio of the basement membrane.

In accordance with the present invention, particular neuronal populations were found to contain high levels of ABCA1 mRNA. For example, cerebellar Purkinje cells had large amounts of ABCA1 mRNA that was restricted to the cell bodies, whereas cerebellar granule neurons did not show any evidence of ABCA1 expression. Cortical and striatal neurons as well as glia contained moderately high levels of ABCA1 mRNA. ABCA1 protein was identified in various tissues using Western Blotting.

Tissues from adult FVB mice were homogenized in an ice-cold buffer containing 20 mM Hepes, 5 mM Kcl, 5 mM MgCl₂, 0.5% (v/v) Triton X-100, and Complete protease inhibitor (Roche). Homogenates were sonicated once for 10 seconds followed by centrifugation at 12,000 rpm for 5 min at 4° C. The protein concentration in tissue or cellular supernatants was determined by the Lowry assay. Equal amounts of protein (typically 80 μg) were separated on 7.5% SDS-PAGE gels and electrophoretically transferred to PVDF membrane (Millipore). Membranes were probed with polyclonal anti-ABCA1 antibody as well as a monoclonal anti-glyceraldehyde phosphate dehydrogenase antibody (Chemicon) as a control for equal loading. Immunoreactivity was detected by ECL (Amersham). Protein abundance was calculated by densitometry using NIH Image software and normalized to GAPDH levels in each tissue. Blots were stripped according to the ECL protocol (Amersham).

A polyclonal antibody, generated against the synthetic peptide corresponding to amino acids 2236-2259 of the human ABCA1 protein, or a monoclonal antibody generated against the second nucleotide binding domain of ABCA1, were used to determine the tissue distribution of ABCA1 protein on Western blots obtained from at least 3 individual wild-type animals. Both antibodies gave identical results. Testis contained high levels of ABCA1 protein, whereas brain contained only moderate amounts. A doublet of ABCA1 was observed in the brain and testis. Both bands were confirmed to be ABCA1 by reprobing the blots with a second ABCA1-specific antibody that recognizes a distinct epitope. The lower molecular weight species may represent an alternative form of ABCA1 generated, for example, by posttranslational modification or alternative splicing. ABCA1 expression was compared between the FVB mice and age matched C57/BI6 mice with no marked differences in the distribution pattern or protein abundance. ABCA1 protein was also identified in the placenta.

As used herein, the genomic sequence of human ABCA1, including exons 1-50, is disclosed in WO 01/15676 (as SEQ ID NO: 1 and FIG. 1 therein). The corresponding human ABCA1 protein is also disclosed in WO 01/15676 as SEQ ID NO: 5 (in FIG. 2A) therein. Human ABCA1 cDNA is disclosed in WO 01/15676 (as SEQ ID NO: 6 and FIG. 2B therein, with FIG. 3 showing a summary of locations in SEQ ID NO: 1). These sequences are also disclosed as SEQ ID NO: 1-3, respectively, in U.S. Provisional Application No. 60/297,102, filed 8 Jun. 2001, and the disclosures of all of which are hereby incorporated by reference in their entirety.

In accordance with the foregoing, the present invention provides a method of identifying an agent useful in regulating fertility in a mammal comprising:
(a) administering to a mammal an agent that modulates ABCA1 biological activity, and
(b) detecting a change in fertility of said mammal following said administering and compared to when said agent is not administered,
thereby identifying an agent useful in regulating fertility of said mammal.

In specific embodiments, such agent may inhibit or enhance the activity of ABCA1 biological activity, wherein the latter may include ABCA1-gene expression or activities of the ABCA1-polypeptide, such as HDL-cholesterol transport, ion transport, ATP binding, ATP hydrolysis, and/or phospholipid transport, and also may include agents that modulate other activities of ABCA1, such as a change in stability of ABCA1 polypeptide, a change in ABCA1 membrane insertion and a change in ABCA1 membrane channel formation.

Such agents may have the effect of either increasing or decreasing fertility in an animal, especially a mammal, most especially a human being. In one such embodiment, the mammal is male and ABCA1-modulating agent has the effect of increasing capacitation of one or more sperm cells of this male and thereby increase fertility. In a preferred embodiment of the latter effect, the agent is a positive modulator of one or more of the functions of ABCA1 genes or proteins and thus has the effect of enhancing ABCA1-biological activity.

In another such embodiment, the mammal is male and the agent decreases capacitation of one or more sperm cells of this male, thereby reducing fertility. In a preferred embodiment of the latter effect, the agent is a negative modulator of one or more of the functions of ABCA1 genes or proteins and thus has the effect of decreasing ABCA1-biological activity.

In another such embodiment, the mammal is female and the agent may increase fertility or decrease fertility. In one such embodiment, the ABCA1-modulating agent promotes implantation into the endometrium, thereby increasing fertility. In a preferred embodiment of the latter process, the agent is a positive modulator of ABCA1 gene expression or polypeptide activity and thus inhibits ABCA1-biological activity.

In another such embodiment, the ABCA1-modulating agent inhibits implantation in the endometrium, thereby decreasing fertility. In a preferred embodiment of the latter process, the agent is a negative modulator of ABCA1 gene expression or polypeptide activity and thus inhibits ABCA1-biological activity.

In a further aspect, the present invention relates to a method for identifying an agent useful in regulating fertility in a male mammal comprising:
(a) contacting a sperm cell of said mammal with an agent that modulates ABCA1 biological activity and under conditions promoting said contacting and supporting viability of said sperm cell, and
(b) detecting a change in the ability of said sperm cell to fertilize an ovum of a mammal of the same species as compared to when said sperm cell is not so contacted,
thereby identifying an agent useful in regulating fertility of said male mammal.

In specific embodiments thereof, the agent may inhibit ABCA1 biological activity or may enhance ABCA1 biological activity, which activity may include gene expression or any of the functions of the ABCA1-polypeptide, including HDL-cholesterol transport, ion transport, ATP binding, ATP hydrolysis, and/or phospholipid transport, or may include effects on stability of ABCA1 polypeptide, a change in ABCA1 membrane insertion and a change in ABCA1 membrane channel formation.

Such agents may increase fertility by increasing the ability of said sperm cell to fertilize said ovum. In a specific embodiment, this increase is due to an increase in capacitation of the sperm cell(s), such as where the agent is a positive modulator of ABCA1-biological activity.

Alternatively, the agent may decrease the ability of said sperm cell to fertilize said ovum, especially where this decrease is due to a decrease in capacitation of said sperm cells, such as where the agent is a negative modulator of ABCA1 biological activity.

In a still further aspect, the present invention relates to a method for regulating fertility in a mammal comprising administering to said mammal an agent having fertility-regulating ability in one or more of the screening assays disclosed herein, especially where said agent was first identified as having such physiological activity using said screening assay.

Where said mammal is male, the agent will preferably modulates fertility by modulating the fertility of the sperm cells of said male mammal, especially where the agent has the effect of regulating capacitation of the sperm cells of said male mammal.

Where the mammal is female, the agent preferably modulates fertility by modulating implantation into the endometrium of said female mammal.

In a still further aspect, the present invention relates to a method for modulating the ability of a sperm cell to fertilize an ovum of an animal of the same species as said sperm cell comprising contacting said sperm cell with an agent that exhibits fertility-regulating ability using a screening method as disclosed herein. In a preferred embodiment, said modulation is an increase in the ability of said sperm cell to fertilize said ovum, especially where this agent acts to promote capacitation of said sperm cells. In an alternative embodiment, said modulation is a decrease in the ability of the sperm cell to fertilize the ovum, especially where this agent acts to inhibit, or prevent, capacitation of said sperm cells.

In a yet still further aspect, the present invention provides a method of treating adverse conditions related to fertility processes, such as adverse conditions arising from pregnancy, especially conditions such as preeclampsia, involving such processes as hypertension and edema resulting from, or related to, pregnancy.

In a preferred embodiment of such processes, the present invention provides a method of treating preeclampsia in a mammal comprising administering to a mammal afflicted therewith an effective amount of an agent that exhibits fertility-regulating ability using a method of claim 28-43.

In another such preferred embodiment, the present invention relates to a method of preventing preeclampsia in a mammal comprising administering to a mammal at risk thereof an effective amount of an agent that exhibits fertility-regulating ability using a screening method disclosed herein, most preferably where said method was used to first identify such agent as having this activity. In a preferred embodiment, the mammal to be treated is a human being.

Cellular Localization of ABCA1 Protein by Immunohistochemistry

Paraffin-embedded tissue sections (3 μm) were mounted on glass slides and deparaffinized in graded alcohols reaching 95%. After a five minute incubation in 1.5% H₂O₂ in methanol, slides were washed in water and rinsed in Tris-buffered saline (pH 7.6). Slides were placed in sodium citrate buffer (pH 6.0) and steamed for thirty minutes. After another water and TBS rinse, the tissues were blocked for thirty minutes in 1:20 normal goat serum. This was followed by incubation with the a polyclonal ABCA1 antibody for 30 minutes in a humidified chamber, washing several times in TBS, and incubation with goat anti-rabbit secondary antibody for forty minutes. Peroxidase conjugated streptavidin was then applied for thirty minutes, with washes in TBS both before and after the incubation. DAB chromagen solution was added for the final detection step. After washing in distilled water, a hematoxylin counterstain was applied and slides were then washed, dehydrated and mounted. Images were recorded using the SPOT Camera system with a Nikon upright microscope. Controls included omission of primary antibody, in which case, no staining was observed, and inclusion of a non-related polyclonal primary antibody, in which case a different pattern of expression was observed.

Expression of ABCA1 protein within individual cells from various tissues was determined using a polyclonal antibody raised against the synthetic peptide corresponding to amino acids 2236-2259 of the human ABCA1 protein. Neuronal populations that expressed ABCA1 mRNA also expressed the protein, however, ABCA1 protein was detected in both the soma and the neuronal arbor of cerebellar Purkinje cells whereas the mRNA that was restricted to the cell bodies. Cortical and striatal neurons as well as glia also contained moderately high levels of ABCA1 protein.

The present invention provides screening assays for ABCA1 modulating agents, such as small organic compounds, that have desired effects on neurological conditions and fertility processes. Useful therapeutic compounds include those that modulate the expression (including stability) or activity of ABCA1. Such methods may be in vitro and involve addition of ABCA1 modulating agents to a medium containing cells whose function represents a model of a disease process or may be in vivo wherein such modulators are administered to an animal, such as an animal model of disease or the like, and the desired physiological effect is noted following such administration and as a result thereof. Such animals may include rodents and mammals, including humans. An extensive list of ABCA1 screening assays is set forth in PCT Publication WO 00/55318, WO 01/15676, and WO 01/32184, as well as elsewhere. The instant invention is directed to screening assays for compounds found to have ABCA1 modulating activity.

In one example, ABCA1 transports interleukin-1β (IL-1β) across the cell membrane and out of cells. IL-1β is a precursor of the inflammatory response and, as such, inhibitors or antagonists of ABCA1 expression or biological activity may be useful treating or preventing an amyloidogenic or non-amyloidogenic disease of the nervous system, traumatic injury to the nervous system, a reproductive defect. In another example, the ability of macrophages to ingest apoptotic bodies is impaired after antibody-mediated blockade of ABCA1. Accordingly, compounds that modulate ABCA1 expression, stability, or biological activity would be useful for the treatment of macrophage-related disorders.

ABCA1 modulators useful in the methods of the present invention include any that affect the expression, stability or activity of ABCA1 at either the gene or protein level. These include agents that affect expression of the ABCA1 gene either directly by binding thereto or by a trans effect on some type of transcription factor that regulates ABCA1 expression. A compound that binds to a promoter or enhancer site that affects ABCA1 expression may also find use. Thus, transcription factors are themselves drug targets.

Transcription factors known to regulate other genes in the regulation of apolipoprotein genes or other cholesterol- or lipid-regulating genes are of particular relevance. Such factors include, but are not limited to, the steroid response element binding proteins (SREBP-1 and SREBP-2), and the PPAR (peroxisomal proliferation-activated receptor), RXR, and LXR transcription factors.

Compounds known to modulate LXR activity include, without limitation, 24-(S),25-epoxycholesterol; 24(S)-hydroxycholesterol; 22-(R)-hydroxycholesterol; 24(R),25-epoxycholesterol; 22(R)-hydroxy-24(S),25-epoxycholesterol; 22(S)-hydroxy-24(R),25-epoxycholesterol; 24-(S),25-iminocholesterol; methyl-38-hydroxycholonate; N,N-dimethyl-3β-hydroxycholonamide; 24(R)-hydroxycholesterol; 22(S)-hydroxycholesterol; 22(R),24(S)-dihydroxycholesterol; 25-hydroxycholesterol; 22(R)-hydroxycholesterol; 22(S)-hydroxycholesterol; 24(S),25-dihydroxycholesterol; 24(R),25-dihydroxycholesterol; 24,25-dehydrocholesterol; 25-epoxy-22(R)-hydroxycholesterol; 20(S)-hydroxycholesterol; (20R,22R)-cholest-5-ene-3β,20,22-triol; 4,4-dimethyl-5-α-cholesta-8,14,24-trien-3-β-ol; 7α-hydroxy-24(S),25-epoxycholesterol; 7β-hydroxy-24(S),25-epoxycholesterol; 7-oxo-24(S),25-expoxycholesterol; 7α-hydroxycholesterol; 7-oxocholesterol; and desmosterol. Additional LXR-modulating compounds are described, for example, in Janowski et al., Nature 383:728-731, 1996; Lehman et al., J. Biol. Chem. 272:3137-3140, 1997; and Janowski et al., Proc. Natl. Acad. Sci. 96:266-271, 1998, each of which is hereby incorporated by reference). In addition one in the art will recognize that synthetic sterols having LXR-modulating activity can be readily identified using screening methods known in the art (see, for example, Janowski et al., Proc. Natl. Acad. Sci. 96:266-271, 1998). Non-steroidal agonists such as RIP140 protein, antibodies (monoclonal or polyclonal) specific for LXR or LXR; tetradecycloxy-furnacarboxylic acid (TOFA;); tetradecylthioacetic acid; as well as other fatty acids (see, for example, Tobin et al. Molec. Endocrin. 14: 741-752, 2000) are also useful LXR-modulating agents.

Compounds that modulate RXR-mediated transcriptional activity will also modulate ABCA1 expression. Numerous RXR-modulating compounds (retinoid compounds) are known in the art, including, for example, heteroethylene derivatives; tricyclic retinoids; trienoic retinoids; benzocycloalkenyl-alka:di- or trienoic acid derivatives; bicyclic-aromatic compounds and their derivatives; bicyclylmethyl-aryl acid derivatives; phenyl-methyl heterocyclic compounds; tetrahydro-napthyl compounds; arylthio-tetrahydro-naphthalene derivatives and heterocyclic analogues; 2,4-pentadienoic acid derivatives; tetralin-based compounds; nonatetraenoic acid derivatives; SR11237; dexamethasone; hydroxy, epoxy, and carboxy derivatives of methoprene; bicyclic benzyl, pyridinyl, thiophene, furanyl, and pyrrole derivatives; benzofuran-acrylic acid derivatives; aryl-substituted and aryl and (3-oxo-1-propenly)-substituted benzopyran, benzothiopyran, 1,2-dihydroquinoline, and 5,6-dihydronaphthalene derivatives; vitamin D3 (1,25-dihydroxyvitamin D3) and analogs; 24-hydroxylase inhibitor; mono-or polyenic carboxylic acid derivatives; tetrahdroquinolin-2-one-6 or 7-yl and related derivatives; tetrahydronaphthalene; oxyiminoalkanoic acid derivatives; LG 100268; and LGD 1069. Additional compounds include BRL 49653; troglitazone; pioglitazone; ciglitazone; WAY-120; englitazone; AD 5075; and darglitazone.

PPARs may alter transcription of ABCA1 by mechanisms including heterodimerization with retinoid X receptors (RXRs) and then binding to specific proliferator response elements (PPREs). Examples of such PPARs include PPARα, β, γ and δ. These distinct PPARs have been shown to have transcriptional regulatory effects on different genes. PPAR is expressed mainly in liver, whereas PPAR is expressed in predominantly in adipocytes. Activation of PPAR results in altered lipoprotein metabolism through PPAR's effect on genes such as lipoprotein lipase (LPL), apolipoprotein CIII (apo CIII) and apolipoprotein AI (apo AI) and AII (apo AII). PPAR activation results in overexpression of LPL and apoA-I and apoA-II, but inhibits the expression of apo CIII. PPAR activation also inhibits inflammation, stimulates lipid oxidation and increases the hepatic uptake and esterification of free fatty acids (FFA's). PPAR and PPAR activation may inhibit nitric oxide (NO) synthase in macrophages and prevent interleukin-1 (IL-1) induced expression of IL-6 and cyclo-oxygenase-2 (COX-2) and thrombin induced endothelin-1 expression secondary to negative transcriptional regulation of NF-KB and activation of protein-1 signaling pathway. It has also been shown that PPAR induces apoptosis in monocyte-derived macrophages through the inhibition of NF-KB activity.

Activation of PPARα can be achieved by compounds such as fibrates, estradiol, arachidonic acid derivatives, WY-14,643 and LTB4 or 8(s)HETE. PPAR activation can be achieved through compounds such as thiozolidinedione antidiabetic drugs, 9-HODE and 13-HODE. Additional compounds such as nicotinic acid or HMG CoA reductase inhibitors may also alter the activity of PPARs.

Compounds which alter activity of any of the PPARs (e.g., PPARα or PPARγ) may have an effect on ABCA1 expression and thereby could affect HDL levels, triglyceride levels, atherosclerosis, and risk of CAD. PPARs are also regulated by fatty acids (including modified fatty acids such as 3 thia fatty acids), leukotrienes such as leukotriene B4 and prostaglandin J2, which is a natural activator/ligand for PPARγ. Drugs that modulate PPARs may therefore have an important effect on modulating lipid levels (including HDL and triglyceride levels) and altering CAD risk. This effect could be achieved through the modulation of ABCA1 gene expression. Drugs may also effect ABCA1 gene expression and thereby ABCA1-mediated cholesterol efflux, by an indirect effect on PPARs via other transcriptional factors such as adipocyte differentiation and determination factor-1 (ADD-1) and sterol regulatory element binding protein-1 and 2 (SREBP-1 and 2). Drugs with combined PPARα and PPARγ agonist activity or PPARα and PPARγ agonists given in combination for example, may increase ABCA1 activity even more.

Additional transcription factors which may also have an effect in modulating ABCA1 gene expression and thereby male fertility and contraception, the risk of non-amyloidogenic diseases of the nervous system and the recovery from acute nervous system injury (ie. traumatic brain injury, traumatic spinal cord injury) include REV-ERBα, SREBP-1 & 2, ADD-1, EBPα, CREB binding protein, P300, HNF 4, RAR, and RORα.

Agents that have been shown to inhibit ABCA1 include, for example, the anti-diabetic agents glibenclamide and glyburide, flufenamic acid, diphenylamine-2-carbonic acid, sulfobromophthalein, and DIDS.

Agents that upregulate ABCA1 expression or biological activity include but are not limited to protein kinase A, protein kinase C, vanadate, okadaic acid, and IBMX1.

Those in the art will recognize that other compounds can also modulate ABCA1 biological activity, and these compounds are also in the spirit of the invention.

As already noted, the ABCA1 modulators for use in the methods of the invention may affect any type of ABCA1-mediated activity and may thus be agents that modulate the expression of the ABCA1 gene as well as affecting activity at the protein level. Among the latter group would be modulators of ATP binding, ATP hydrolysis, and/or phospholipid transport, ion transport across a membrane, cholesterol transport across a membrane, stability of the ABCA1 protein within the plasma membrane, insertion of the ABCA1 protein into a cellular membrane and also agents that modulate the ability of ABCA1 to form channels in a membrane.

By way of non-limiting example, mutant ABCA1 polypeptides are likely to have dominant negative activity (e.g., activity that interferes with wild-type ABCA1 function). An assay for a compound that can interfere with such a mutant may be based on any method of quantitating normal ABCA1 activity in the presence of the mutant. For example, normal ABCA1 facilitates cholesterol efflux, and a dominant negative mutant would interfere with this effect. The ability of a compound to counteract the effect of a dominant negative mutant may be based on cellular cholesterol efflux, or on any other normal activity of the wild-type ABCA1 that is reduced in the mutant. Such agents may find use in the methods of the invention.

Compounds of the invention, including but not limited to, ABCA1 polypeptides, ABCA1 nucleic acids, other ABC transporters, LXR-modulating compounds, RXR-modulating compounds, and any therapeutic agent that modulates biological activity or expression of ABCA1 identified using any of the methods disclosed herein, may be administered with a pharmaceutically-acceptable diluent, carrier, or excipient, in unit dosage form. Conventional pharmaceutical practice may be employed to provide suitable formulations or compositions to administer such compositions to patients. Any appropriate route of administration may be employed, for example, intravenous, perenteral, subcutaneous, intramuscular, intracranial, intraorbital, ophthalmic, intraventricular, intracapsular, intraspinal, intracisternal, intraperitoneal, intranasal, aerosol, or oral administration. Therapeutic formulations may be in the form of liquid solutions or suspension; for oral administration, formulations may be in the form of tablets or capsules; and for intranasal formulations, in the form of powders, nasal drops, or aerosols.

Methods well known in the art for making formulations are found in, for example, Remington: The Science and Practice of Pharmacy, (19th ed.) ed. A. R. Gennaro AR., 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agonists of the invention include ethylenevinyl acetate copolymer particles, osmotic pumps, implantable infusion systems, and liposomes. Formulations for inhalation may contain excipients, or example, lactose, or may be aqueous solutions containing, for example, polyoxyethylene-9-lauryl ether, glycocholate and deoxycholate, or may be oily solutions for administration in the form of nasal drops, or as a gel.

In general, novel drugs for treating or preventing a neurological disease or disorder, or for modulating an anaesthetic or a fertility process, are identified from large libraries of both natural product or synthetic (or semi-synthetic) extracts or chemical libraries according to methods known in the art. Those skilled in the field or drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their ability to modulate ABCA1 gene expression or ABCA1 biological activity should be employed whenever possible.

When a crude extract is found to modulate ABCA1 gene expression, ABCA1 biological activity, or a combination thereof, further fractionation of the positive lead extract is necessary to isolate chemical constituent responsible for the observed effect. Thus, the goal of the extraction, fractionation, and purification process is the careful characterization and identification of a chemical entity within the crude extract having the ability to modulate ABCA1 gene expression or activity. The same in vivo and in vitro assays described herein for the detection of activities in mixtures of compounds can be used to purify the active component and to test derivatives thereof. Methods of fractionation and purification of such heterogeneous extracts are known in the art. If desired, compounds shown to be useful agents for the treatment of pathogenicity are chemically modified according to methods known in the art. Compounds identified as being of potential therapeutic value are subsequently analyzed using any standard animal models known in the art.

It is understood that compounds which modulate the activity of proteins that modulate ABCA1 gene expression or activity are useful compounds for the purposes described herein. Exemplary compounds are provided herein; others are known in the art.

Compounds that are structurally related to cholesterol, or that mimic ApoAI or a related apolipoprotein, and increase ABCA1 biological activity are particularly useful compounds in the invention. Other compounds, known to act on the MDR protein, can also be used or derivatized and assayed for their ability to increase ABCA1 biological activity. Exemplary MDR modulators are PSC833, bromocriptine, and cyclosporin A. Other examples of compounds that may be assayed for the ability to increase ABCA1 biological activity include oxysterols and their derivatives.

In conjunction with such treatment, the pharmacogenomics (e.g., the study of the relationship between an individual's genotype and that individual's response to a foreign compound or drug) of the individual may be considered. Differences in efficacy of therapeutics can lead to severe toxicity or therapeutic failure by altering the relation between dose and blood concentration of the pharmacologically active drug. Thus, the pharmacogenomics of the individual permits the selection of effective agents for prophylactic or therapeutic treatments based on a consideration of the individual's genotype. Such pharmacogenomics can further be used to determine appropriate dosages and therapeutic regimens. Accordingly, the activity of ABCA1 protein, expression of ABCA1 nucleic acid, or mutation content of ABCA1 genes in an individual can be determined to thereby select appropriate agent(s) for therapeutic or prophylactic treatment of the individual. Pharmacogenomics deals with clinically significant hereditary variations in the response to drugs due to altered drug disposition and abnormal action in affected persons (Eichelbaum, M., Clin. Exp. Pharmacol. Physiol., 23:983-985, 1996; Linder, M. W., Clin. Chem., 43:254-266, 1997). In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). Altered drug action may occur in a patient having a polymorphism (e.g., an single nucleotide polymorphism or SNP) in promoter, intronic, or exonic sequences of ABCA1. Thus by determining the presence and prevalence of polymorphisms allow for prediction of a patient's response to a particular therapeutic agent. In particular, polymorphisms in the promoter region may be critical in determining the risk and/or amenability to treatment of a neurological disease or disorder, or for modulating an anaesthetic or a fertility process.

A number of polymorphisms have been located in the 5′-UTR and 5′-regulatory regions of the ABCA1-gene and represent valuable pharmacogenomic targets. These have been published in WO 01/15676 (published 8 Mar. 2001).

More specifically, several polymorphisms in the 5′ regulatory region of human ABCA1 have been identified (with reference to the sequence of the ABCA1-gene as disclosed in WO 01/15676 (published 8 Mar. 2001), the disclosure of which is hereby incorporated in its entirety. Because of the location of such polymorphisms, it is likely that ABCA1 gene expression will differ among humans having different promoter polymorphisms, and these individuals may also respond differently to the same drug treatment. Thus, using these newly-identified polymorphisms, one can tailor drug treatment depending on which polymorphism(s) is/are present in a patient.

The following polymorphisms have been previously examined for their effect on cholesterol regulation and the predisposition for the development of cardiovascular disease. The polymorphisms may also be associated with neurological diseases or disorders, or defects of a fertility process. The polymorphisms are numbered from the nucleotide described as position 1 (Pullinger et al., supra), naming the first exon number 1.

Substitution of A for G at nucleotide 1051 (R219K). Such notation means that an R (or arginine) at residue number 219 has been replaced by a K (or lysine) due to the polymorphism in the gene at nucleotide 1051. Carriers of this variant have reduced triglyceride levels, increased HDL cholesterol levels (particularly in younger individuals), and reduced CAD (U.S. patent application Ser. No. 09/654,323; PCT/IB00/01492).

Substitution of C for T at nucleotide 1591 (V399A). This variant was associated with a trend towards increased HDL cholesterol in carriers and fewer coronary events.

Substitution of A for G at nucleotide 2706 (V771 M). Carriers of this variant have been shown to have decreased CAD.

Substitution of C for A at nucleotide 2715 (T774P). This variant was seen less often in individuals with low HDL cholesterol levels or CAD than in controls.

Substitution of C for G at nucleotide 2723 (K776N). This variant has been found at a lower frequency (0.54% versus 1.89%) in a coronary artery disease population versus a control population of similar Dutch background.

Substitution of T for C at nucleotide 3120 (R909X). Carriers of this variant have a trend to decreased HDL levels.

Substitution of C for T at nucleotide 3667 (M1091T). Carriers of this variant have a severe impairment of cholesterol efflux and reduced HDL levels. Homozygotes have splenomegaly alone or in association with coronary artery disease.

Substitution of C for G at nucleotide 3911 (E1172D). This variant is seen at lower frequencies in individuals with low HDL and in some populations with premature coronary artery disease.

Substitution of A for G at nucleotide 5155 (R1587K). This variant is associated with decreased HDL cholesterol levels in carriers.

Substitution of G for C at nucleotide 5587 (S1731C). Two FHA individuals who have this variant on the other allele have much lower HDL cholesterol (0.155 Å0.025) than the FHA individuals in the family who do not have this variant on the other allele (0.64 Å0.14, p=0.0009). This variant has also been found in one general population French Canadian control with HDL at the 8^{th} percentile (0.92) and one French Canadian individual from a population selected for low HDL cholesterol levels and coronary disease (0.72).

Substitution of G for A at nucleotide 2723 (I883M). This variant has been seen at a much higher frequency in individuals of Dutch ancestry with premature coronary artery disease. Furthermore, homozygous carriers of this variant have significantly increased CAD progression compared to non-carriers.

Substitution of A for G at nucleotide 2868 (V825I). Carriers of this variant had significantly more CAD events than individuals who do not have this variant.

Substitution of C for G at nucleotide −191. Homozygous carriers of this variant have a three-fold increase in the frequency of coronary events (33.3% versus 11.2%, p=0.003) and a nearly double frequency of a positive family history of CAD (73.3% versus 47.7%, p=0.01).

Substitution of G for C at nucleotide −17. Carriers of this variant have significantly decreased coronary events (12.3% versus 18.2%, p=0.04) and a significantly decreased incidence of myocardial infarction (heart attack, 43.6% versus 52.8%, p=0.02).

Substitution of T for C at nucleotide 69. Carriers of this variant have increased CAD progression compared to non-carriers.

Substitution of G for C at nucleotide 117. Carriers of this variant have a trend towards decreased progression of CAD compared to non-carriers.

Insertion of CCCT at nucleotide −1163 in intron 1. Carriers of this variant have a trend to lower HDL cholesterol levels.

Substitution of G for A at nucleotide −1095 in intron 1. Homozygous carriers of this variant have a trend towards decreased HDL cholesterol and increased triglyceride levels compared to non-carriers.

Substitution of A for G at nucleotide −1027 in intron 1. Carriers of this variant are also carriers of the G(−720)A. Thus the effects attributed to that variant may also be attributed to carriers of this variant.

Substitution of A for G at nucleotide −720 in intron 1. Homozygous carriers of this variant had a trend towards an increased frequency of a positive family history of myocardial infarction.

Substitution of C for A at nucleotide −461 in intron 1. Carriers of this variant are also carriers of the A(−362)G. Thus the effects attributed to that variant may also be attributed to carriers of this variant.

Substitution of G for A at nucleotide −362 in intron 1. Carriers of this variant have decreased triglyceride levels compared to non-carriers.

Insertion of G at nucleotide 319. Carriers of this variant have increased CAD compared to non-carriers.

Substitution of G for C at nucleotide 378. Carriers of this variant are also carriers of the InsG319. Thus the effects attributed to that variant may also be attributed to carriers of this variant.

In addition to the mutations in the ABCA1 gene described herein, we have detected polymorphisms in the human ABCA1 gene (FIG. 4). These polymorphisms are located in promoter, intronic, and exonic sequence of ABCA1. Using standard methods, such as direct sequencing, PCR, SSCP, or any other polymorphism-detection system, one could easily ascertain whether these polymorphisms are present in a patient prior to the establishment of a drug treatment regimen for a patient diagnosed as having or being at increased risk for a neurological disease or disorder, or a defect of a fertility process or any other ABCA1-mediated condition. It is possible that some these polymorphisms are, in fact, weak mutations and individuals harboring such mutations may have an increased risk for these conditions. Thus, these polymorphisms may also be useful in diagnostic assays.

Because of its link to diverse physiological processes, ABCA1 is useful as a general assay for therapeutic agents. Thus, the present invention also provides for an ABCA1 assay as a basis for identifying therapeutic agents, such as those useful in modulating fertility and in treating neurological diseases and disorders.

In one such aspect, the present invention provides a method to identify a therapeutic agent for modulating fertility in a mammal comprising
(a) providing an assay which measures a biological activity of ABCA1,
(b) contacting said assay with a compound, and
(c) measuring whether said compound modulates said biological activity of ABCA1,
wherein a compound which modulates said biological activity of ABCA1 is thereby identified as said therapeutic agent for modulating fertility in a mammal.

In a preferred embodiment thereof, the therapeutic agent is a contraceptive. In another preferred embodiment, said therapeutic agent promotes male fertility. Thus, the present invention also provides a method of modulating fertility in a mammal comprising administering to said mammal a compound which modulates the biological activity of ABCA1. Preferably, such compounds are identified using the corresponding screening assay. In addition, such agents preferably inhibit an ABCA1-dependent biological activity.

The present invention also provides a method to identify a therapeutic agent for treating a neurological disease or disorder in a mammal comprising
(a) providing an assay which measures a biological activity of ABCA1,
(b) contacting said assay with a compound, and
(c) measuring whether said compound modulates said biological activity of ABCA1,
wherein a compound which modulates said biological activity of ABCA1 is thereby identified as said therapeutic agent for treating a neurological disease or disorder in a mammal.

Such agents may enhance or inhibit an ABCA1-dependent biological activity.

In accordance therewith, the present invention also contemplates a method of treating a neurological disease or disorder in a mammal comprising administering to said mammal a compound which modulates the biological activity of ABCA1, especially where said compound was identified according to the screening methods disclosed herein.

The present invention also relates to a process that comprises a method for producing a product comprising identifying an agent according to one of the disclosed processes for identifying such an agent (i.e., the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after performing the screening process, using one or more assay processes of the present invention, then imparts to a second user (user 2), verbally or in writing or some equivalent fashion, sufficient information to identify the compounds having a particular modulating activity (for example, the code number with the corresponding results). This transmission of information from user 1 to user 2 is specifically contemplated by the present invention.

1. A method for identifying an agent useful in treating an ABCA1-dependent neurological condition comprising:
(a) administering to an animal exhibiting said neurological condition an agent that modulates ABCA1 biological activity, and
(b) detecting a beneficial change in said neurological condition in said animal following said administering and compared to when said agent is not administered,
thereby identifying an agent useful in treating said ABCA1-dependent neurological condition.

2. The method of claim 1 wherein said agent inhibits ABCA1 biological activity.

3. The method of claim 1 wherein said agent enhances ABCA1 biological activity.

4. The method of claim 1 wherein said ABCA1 biological activity is ABCA1-gene expression.

5. The method of claim 1 wherein said ABCA1 biological activity is selected from the group consisting of HDL-cholesterol transport, ion transport, ATP binding, ATP hydrolysis, and phospholipid transport.

6. The method of claim 1 wherein modulation of ABCA1 biological activity is due to a process selected from the group consisting of a change in stability of ABCA1 polypeptide, a change in ABCA1 membrane insertion and a change in ABCA1 membrane channel formation.

7. The method of claim 1 wherein said neurological condition is a member selected from the group consisting of a neurological condition of the central nervous system and a neurological condition of the peripheral nervous system.

8. The method of claim 7 wherein said neurological condition is characterized by neuronal loss or dysfunction.

9. The method of claim 7 wherein said neurological condition is a member selected from the group consisting of Alzheimer's Disease, dementia pugilistica, Parkinson's Disease, Huntington's Disease, Niemann-Pick disease, multiple sclerosis, a neuropathy and an ischemic condition.

10. The method of claim 9 wherein said ischemic condition is an ischemic condition of the central, peripheral, or compression type.

11. The method of claim 9 wherein said ischemic condition is a member selected from the group consisting of stroke and cerebral artery infarction.

12. The method of claim 7 wherein said neurological condition is caused by a defect in myelin repair or production.

13. The method of claim 7 wherein said defect in myelin repair or production is caused by a process selected from the group consisting of demyelination, the removal of myelin debris following injury and remyelination.

14. A method of treating a neurological condition comprising administering to an animal afflicted with such condition an effective amount of an agent that exhibits beneficial activity in a method of claim 1.

15. The method of claim 14 wherein said agent inhibits ABCA1 biological activity.

16. The method of claim 14 wherein said agent enhances ABCA1 biological activity.

17. The method of claim 14 wherein said ABCA1 biological activity is ABCA1-gene expression.

18. The method of claim 14 wherein said ABCA1 biological activity is selected from the group consisting of HDL-cholesterol transport, ion transport, ATP binding, ATP hydrolysis, and phospholipid transport.

19. The method of claim 14 wherein modulation of ABCA1 biological activity is due to a process selected from the group consisting of a change in stability of ABCA1 polypeptide, a change in ABCA1 membrane insertion and a change in ABCA1 membrane channel formation.

20. The method of claim 14 wherein said neurological condition is a member selected from the group consisting of a neurological condition of the central nervous system and a neurological condition of the peripheral nervous system.

21. The method of claim 20 wherein said neurological condition is characterized by neuronal loss or dysfunction,

22. The method of claim 21 wherein said neurological condition is a member selected from the group consisting of Alzheimer's Disease, dementia pugilistica, Parkinson's Disease, Huntington's Disease, Niemann-Pick disease, multiple sclerosis, a neuropathy and an ischemic condition.

23. The method of claim 22 wherein said ischemic condition is an ischemic condition of the central, peripheral, or compression type.

24. The method of claim 22 wherein said ischemic condition is a member selected from the group consisting of stroke and cerebral artery infarction.

25. The method of claim 20 wherein said neurological condition is caused by a defect in myelin repair or production.

26. The method of claim 20 wherein said defect in myelin repair or production is caused by a process selected from the group consisting of demyelination, the removal of myelin debris following injury and remyelination.

27. The method of claim 14 wherein said agent was first identified as having beneficial activity using a method of claim 1.

28. A method for identifying an agent useful in regulating fertility in a mammal comprising:
(a) administering to a mammal an, agent that modulates ABCA1 biological activity, and
(b) detecting a change in fertility of said mammal following said administering and compared to when said agent is not administered,
thereby identifying an agent useful in regulating fertility of said mammal.

29. The method of claim 28 wherein said agent inhibits ABCA1 biological activity.

30. The method of claim 28 wherein said agent enhances ABCA1 biological activity.

31. The method of claim 28 wherein said ABCA1 biological activity is ABCA1-gene expression.

32. The method of claim 28 wherein said ABCA1 biological activity is selected from the group consisting of HDL-cholesterol transport, ion transport, ATP binding, ATP hydrolysis, and phospholipid transport.

33. The method of claim 28 wherein modulation of ABCA1 biological activity is due to a process selected from the group consisting of a change in stability of ABCA1 polypeptide, a change in ABCA1 membrane insertion and a change in ABCA1 membrane channel formation.

34. The method of claim 28 wherein said agent increases fertility.

35. The method of claim 28 wherein said agent decreases fertility.

36. The method of claim 28 wherein said mammal is male.

37. The method of claim 34 wherein said mammal is male and said agent increases capacitation of a sperm cell of said male.

38. The method of claim 35 wherein said mammal is male and said agent decreases capacitation of a sperm cell of said male.

39. The method of claim 28 wherein said mammal is female.

40. The method of claim 39 wherein said agent increases fertility.

41. The method of claim 39 wherein said agent decreases fertility.

42. The method of claim 40 wherein said agent promotes endometrial implantation.

43. The method of claim 40 wherein said agent interferes with endometrial implantation.

44. A method for identifying an agent useful in regulating fertility in a male mammal comprising:
(a) contacting a sperm cell of said mammal with an agent that modulates ABCA1 biological activity and under conditions promoting said contacting and supporting viability of said sperm cell, and
(b) detecting a change in the ability of said sperm cell to fertilize an ovum of a mammal of the same species as compared to when said sperm cell is not so contacted,
thereby identifying an agent useful in regulating fertility of said male mammal.

45. The method of claim 44 wherein said agent inhibits ABCA1 biological activity.

46. The method of claim 44 wherein said agent enhances ABCA1 biological activity.

47. The method of claim 44 wherein said ABCA1 biological activity is ABCA1-gene expression.

48. The method of claim 44 wherein said ABCA1 biological activity is selected from the group consisting of HDL-cholesterol transport, ion transport, ATP binding, ATP hydrolysis, and phospholipid transport.

49. The method of claim 44 wherein modulation of ABCA1 biological activity is due to a process selected from the group consisting of a change in stability of ABCA1 polypeptide, a change in ABCA1 membrane insertion and a change in ABCA1 membrane channel formation.

50. The method of claim 44 wherein said agent increases the ability of said sperm cell to fertilize said ovum.

51. The method of claim 50 wherein said increase is due to an increase in capacitation of said sperm cells.

52. The method of claim 44 wherein said agent decreases the ability of said sperm cell to fertilize said ovum.

53. The method of claim 52 wherein said decrease is due to a decrease in capacitation of said sperm cells.

54. A method for regulating fertility in a mammal comprising administering to said mammal an agent having fertility-regulating ability using a method of claim 28.

55. The method of claim 54 wherein said regulating is an increase in ABCA1-biological activity.

56. The method of claim 54 wherein said regulation is a decrease in ABCA1-biological activity.

57. The method of claim 54 wherein said agent was first identified as having fertility-regulating ability using a method of claim 54.

58. The method of claim 54 wherein said mammal is male.

59. The method of claim 58 wherein said agent modulates said fertility by modulating the fertility of the sperm cells of said male mammal.

60. The method of claim 54 wherein said mammal is female.

61. The method of claim 60 wherein said agent modulates said fertility by modulating implantation into the endometrium of said male mammal.

62. A method for modulating the ability of a sperm cell to fertilize an ovum of an animal of the same species as said sperm cell comprising contacting said sperm cell with an agent that exhibits fertility-regulating ability using a method of claim 44.

63. The method of claim 62 wherein said modulation is an increase in the ability of said sperm cell to fertilize said ovum.

64. The method of claim 62 wherein said modulation is a decrease in the ability of said sperm cell to fertilize said ovum.

65. The method of claim 64 wherein said agent decreases or prevents capacitation of said sperm cell.

66. The method of claim 62 wherein said modulation is an increase in the ability of said sperm cell to fertilize said ovum.

67. The method of claim 64 wherein said agent promotes capacitation of said sperm cell.

68. A method of treating preeclampsia in a mammal comprising administering to a mammal afflicted therewith an effective amount of an agent that exhibits fertility-regulating ability using a method of claim 28.

69. A method of preventing preeclampsia in a mammal comprising administering to a mammal at risk thereof an effective amount of an agent that exhibits fertility-regulating ability using a method of claim 28.

70. The method of claim 68 or 69 wherein said mammal is a human being.

71. The method of claim 68, 69 or 70 wherein said agent was first identified as having fertility-regulating activity using a method of claim 28.

72. A method for producing a product comprising identifying an agent according to the process of claim 1, 28 or 44 wherein said product is the data collected with respect to said agent as a result of said process and wherein said data is sufficient to convey the chemical structure and/or properties of said agent.

73. A screening method for identifying an agent useful in treating an ABCA1-dependent neurological condition comprising:
(a) administering to an animal exhibiting said neurological condition an agent that modulates ABCA1 biological activity,
(b) detecting a beneficial change in said neurological condition in said animal following said administering as compared to when said agent is not administered, and
thereby identifying an agent useful in treating an ABCA1-dependent neurological condition.

74. The screening method of claim 73 wherein said ABCA1-modulating agent was shown to modulate ABCA1-biological activity prior to use in said screening method.

75. The screening method of claim 73 wherein said ABCA1-modulating agent was shown to modulate ABCA1-biological activity after use in said screening method.

76. A method of treating an animal for an ABCA1-dependent neurological condition comprising administering to an animal afflicted with such condition an effective amount of an ABCA1-modulating agent.

77. The method of claim 76 wherein said agent has activity using a screening method of claim 73.

78. The method of claim 76 wherein said agent was first identified as useful in treating said neurological condition using a screening method of claim 73.

79. The method of claim 76 wherein said agent was not shown to have ABCA1-modulating activity prior to use in said treating method.

80. The method of claim 76 wherein said agent was shown to have ABCA1-modulating activity prior to use in said treating method.

81. A screening method for identifying an agent useful in negating the malfunctioning of a nervous system cell comprising:
(a) contacting a malfunctioning nervous system cell, wherein said malfunctioning promotes the presence of said neurological condition, with an ABCA1-modulating agent under conditions promoting said contacting and otherwise supporting the normal functioning of said cell,
(b) determining a beneficial change in one or more functions of said cell after said contacting wherein said beneficial change is not determined when said contacting does not occur, and
thereby identifying an agent useful in negating malfunctioning of a neurological condition.

82. A screening method for identifying an agent useful in treating an ABCA1-dependent neurological condition comprising:
(a) contacting a malfunctioning nervous system cell, wherein said malfunctioning promotes the presence of said neurological condition, with an ABCA1-modulating agent under conditions promoting said contacting and otherwise supporting the normal functioning of said cell,
(b) determining a beneficial change in one or more functions of said cell after said contacting wherein said beneficial change is not determined when said contacting does not occur, and
thereby identifying an agent useful in treating said neurological condition.

83. A screening method for identifying an agent useful in promoting myelin production in a connective tissue cell whose normal function includes myelin production, comprising:
(a) contacting said connective tissue cell with an ABCA1-modulating agent under conditions promoting said contacting and otherwise supporting myelin production by said cell,
(b) determining an increase in myelin production by said cell after said contacting wherein said increase is not determined when said contacting does not occur, and
thereby identifying an agent useful in promoting myelin production by said cell.

84. The screening method of claim 83 wherein the cell of step (a) is deficient in myelin production.

85. The screening method of claim 83 wherein said cell is found in the central nervous system or the peripheral nervous system.

86. The method of claim 83 wherein said cell is a Schwann cell of an oligodendrocyte.

87. The screening method of claim 81, 82 or 83 wherein said contacting occurs in vitro.

88. The screening method of claim 81, 82 or 83 wherein said contacting occurs in vivo.

89. The screening method of claim 81, 82 or 83 wherein said ABCA1-modulating agent was shown to modulate ABCA1-biological prior to use in said screening method.

90. The screening method of claim 81, 82 or 83 wherein said ABCA1-modulating agent was not shown to modulate ABCA1-biological prior to use in said screening method.

91. A method of treating a neurological condition in an animal comprising administering to an animal afflicted with said condition an effective amount of an agent first identified as having therapeutic activity using a screening method of claim 81, 82 or 83.

92. A method for identifying an ABCA1-related cause of reduced fertility in a male patient afflicted with said reduced fertility comprising identifying in one or more sperm cells from said patient a reduced ABCA1-biological activity relative to a sperm cell from a patient without said ABCA1-related infertility.

93. The method of claim 92 wherein said reduced ABCA1-biological activity is a decrease in activity of an ABCA1-polypeptide in said one or more sperm cells.

94. The method of claim 92 wherein said reduced ABCA1-biological activity is a decreased amount of ABCA1-polypeptide in said one or more sperm cells.

95. The method of claim 92 wherein said reduced ABCA1-biological activity is a decreased expression of an ABCA1 gene in said one or more sperm cells.

96. The method of claim 95 wherein said decreased expression is due to a polymorphism in a promoter or other non-coding region of said ABCA1 gene.

97. The method of claim 92 wherein said reduced ABCA1-biological activity is due to a polymorphism in a coding region of an ABCA1 gene in said one or more sperm cells.

98. A method for identifying a male patient afflicted with reduced fertility as a candidate for treatment of said reduced fertility using an ABCA1-modulating agent comprising identifying in said male patient a reduced amount of ABCA1-biological activity using the method of claim 92.

99. The method of claim 98 wherein said ABCA1-modulator is a positive modulator of ABCA1-biological activity.

100. The method of claim 98 wherein said ABCA1-modulating agent was previously shown to modulate ABCA1-biological activity.

101. The method of claim 98 wherein said ABCA1-modulating agent was not previously shown to modulate ABCA1-biological activity.

102. A method for preventing capacitation of sperm cells during freezing and/or storage comprising freezing and/or storing sperm cells in a composition comprising an inhibitor of ABCA1 biological activity, thereby preventing or retarding capacitation during such freezing and/or storing.

103. A method for facilitating a process requiring sperm fertility, comprising addition of a positive ABCA1 modulator to a sample of sperm cells either not capacitated, or inadequately capacitated, or known to be comprised as to capacitation.

104. The method of claim 102 wherein said process to be facilitated is in vitro fertilization.

105. The method of claim 102 wherein said not capacitated, or inadequately capacitated, or known to be comprised as to capacitation is due to freezing and/or storage of said sperm cells.

106. A method to identify a therapeutic agent for modulating fertility in a mammal comprising
(a) providing an assay which measures a biological activity of ABCA1,
(b) contacting said assay with a compound, and
(c) measuring whether said compound modulates said biological activity of ABCA1,
wherein a compound which modulates said biological activity of ABCA1 is thereby identified as said therapeutic agent for modulating fertility in a mammal.

107. The method of claim 106 wherein said therapeutic agent is a contraceptive.

108. The method of claim 106 wherein said therapeutic agent promotes male fertility.

109. A method of modulating fertility in a mammal comprising administering to said mammal a compound which modulates the biological activity of ABCA1.

110. The method of claim 109 wherein said compound was identified according to the method of claim 105.

111. The method of claim 106 wherein said therapeutic agent enhances an ABCA1-dependent biological activity.

112. The method of claim 106 wherein said therapeutic agent inhibits an ABCA1-dependent biological activity.

113. A method to identify a therapeutic agent for treating a neurological disease or disorder in a mammal comprising
(a) providing an assay which measures a biological activity of ABCA1,
(b) contacting said assay with a compound, and
(c) measuring whether said compound modulates said biological activity of ABCA1,
wherein a compound which modulates said biological activity of ABCA1 is thereby identified as said therapeutic agent for treating a neurological disease or disorder in a mammal.

114. The method of claim 113 wherein said therapeutic agent enhances an ABCA1-dependent biological activity.

115. The method of claim 113 wherein said therapeutic agent inhibits an ABCA1-dependent biological activity.

116. A method of treating a neurological disease or disorder in a mammal comprising administering to said mammal a compound which modulates the biological activity of ABCA1.

117. The method of claim 116 wherein said compound was identified according to the method of claim 113.
